SEQUENCE LISTING (8 sequences)

SEQ ID NO: 1 - protein, 118 aa, Homo sapiens
EVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 2 - protein, 107 aa, Homo sapiens
GQIVLTQSPAIMSASPGEKVTMTCSASSSIDYIHWYQQKSGTSPKRWIYDTSKLASGVPARFSGSGSGTSYSLTISSMEPEDAATYYCHQRNSYPWTFGGGTRLEIR

SEQ ID NO: 3 - protein, 202 aa, Homo sapiens
EEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVRVRRVA

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 118 aa, synthetic construct
EVQLQQSGPELVKPGASMKISCKASGHSFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 6 - protein, 118 aa, synthetic construct
EVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYAYSLDYWGQGTSVTVSS

SEQ ID NO: 7 - protein, 118 aa, synthetic construct
EVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYHSLDYWGQGTSVTVSS

SEQ ID NO: 8 - protein, 107 aa, synthetic construct
GQIVLTQSPAIMSASPGEKVTMTCSASSSIDYIHWYQQKSGTSPKRWIYDTSKLASGVPARFSGSGSGTSYSLTISSMEPEDAATYYCHQRNSAPWTFGGGTRLEIR

(19) United States
(12) Patent Application Publication
(10) Pub. No.: US 2024/0317877 A1
(43) Pub. Date: Sep. 26, 2024

(54) ANTI-HUMAN TRANSFERRIN RECEPTOR ANTIBODY HAVING IMPROVED BLOOD-BRAIN-BARRIER PERMEABILITY, AND MULTI-SPECIFIC ANTIBODY AND PHARMACEUTICAL COMPOSITION WHICH USE SAME

(71) Applicant: (IUCF-HYU (INDUSTRY-UNIVERSITY COOPERATION FOUNDATION HANYANG UNIVERSITY)), Seoul (KR)

(72) Inventors: Seong Eon RYU, Seoul (KR); Hye Hyeon JANG, Seoul (KR); Myeongbin KIM, Seoul (KR); So Hee KIM, Seongnam-si, Gyeonggi-do (KR); Dabin SONG, Gunpo-si, Gyeonggi-do (KR); Sung Ho PARK, Daejeon (KR)

(21) Appl. No.: 18/578,749

(22) PCT Filed: Jul. 15, 2022
(86) PCT No.: PCT/KR2022/010337

§ 371 Date: Mar. 12, 2024

(30) Foreign Application Priority Data
Jul. 15, 2021 (KR) 10-2021-0092822
Jan. 25, 2022 (KR) 10-2022-0010867
Jul. 12, 2022 (KR) 10-2022-0085650

(51) Int. Cl. C07K 16/28; A61K 39/00; A61K 47/68

(52) U.S. Cl. CPC C07K 16/2881; A61K 47/6803; A61K 2039/505; C07K 2317/24; C07K 2317/31; C07K 2317/52; C07K 2317/56

(57) ABSTRACT

An anti-human transferrin receptor antibody having improved blood-brain barrier permeability has a regulated binding affinity to the human transferrin receptor, and thus exhibits excellent blood-brain barrier permeability. Therefore, if the anti-human transferrin receptor antibody is used in a multi-specific antibody or an antibody-drug conjugate, proteins or drug compounds for treating brain diseases can be effectively delivered to brain tissues.

### TECHNICAL FIELD

The present invention relates to an anti-human transferrin receptor antibody having improved blood-brain barrier permeability, and a multi-specific antibody and a pharmaceutical composition using the same, and specifically, to an anti-human transferrin receptor antibody having improved blood-brain barrier permeability through its regulated binding affinity to a human transferrin receptor, and a multi-specific antibody and a pharmaceutical composition that can be effectively applied to the prevention or treatment of brain diseases using the same.

### BACKGROUND ART

The blood-brain-barrier (BBB) is a barrier that exists between the brain and blood vessels, and plays an important role in protecting the regulatory function of the central nervous system from pathogens and potentially dangerous substances by selectively permeating only the nutrients and metabolites necessary for the brain. However, since this selective permeability of the blood-brain barrier blocks the entry of therapeutic agents for brain diseases, there is a problem that many therapeutic agents for brain diseases cannot be delivered to target cells, thereby making it difficult to show clinical effectiveness.

In this regard, research has been published showing that when immunoglobulin (IgG) is injected, only a very low percentage (about 0.1%) is able to penetrate into the central nervous system (CNS) compartment (see Felgenhauer, Klinische Wochenschrift 52, 1158-1164 (1974)). In addition, even in the case of small molecule therapeutic agents, it has been reported that only some drugs with high lipid solubility and very low molecular weight are able to penetrate the blood-brain barrier. As such, since the effectiveness of therapeutic substances is limited by the blood-brain barrier, it is very important to improve blood-brain barrier permeability in the development of therapeutic agents for brain diseases, and various strategies have been attempted for this purpose.

In the case of small molecule therapeutic agents, a method of designing derivatives capable of improving their blood-brain barrier permeability, such as modifying them to have hydrophobic properties, may be used. For example, Korean Patent Application Laid-Open No. 10-2019-0045101 discloses a technology to improve therapeutic efficiency and blood-brain barrier permeability by designing a structurally modified aminophenyl thiazole derivative of T16Ainh-A01, a calcium-dependent chloride channel blocking agent.

Meanwhile, since it is difficult for general lipid-soluble small molecule therapeutic agents to be effective in brain diseases such as Alzheimer's disease and Parkinson's disease, protein or antibody therapeutic agents are being developed. However, since it is impossible for protein or antibody therapeutic agents to directly penetrate the cell membrane of the blood-brain barrier, methods have been attempted to use the transcytosis pathway through endogenous receptors present in cells forming the blood-brain barrier. Antibodies that bind to endogenous receptors present in the blood-brain barrier may traverse the blood-brain barrier by entering cells together with the receptors through endocytosis and exiting on the other side through exocytosis. In this case, by attaching a therapeutic protein or the antigen-binding region of a therapeutic antibody to an antibody, such therapeutic substance may be designed to enter the brain.

In the present invention, as part of the transcytosis strategy through the endogenous receptor, a strategy using an anti-transferrin receptor (TfR) antibody is proposed. The anti-transferrin receptor antibody is an antibody that binds to a transferrin receptor, which is present in large quantities in the blood-brain barrier, and has the characteristics of tightly binding to the receptor and allowing endocytosis to occur easily, but not being easily separated from the receptor when exocytosis occurs. As a result, there is a problem that the anti-transferrin receptor antibody cannot enter the brain and remains attached to the blood-brain barrier cells, thereby reducing the delivery efficiency of the therapeutic substances.

Under the circumstances, the present inventors have attempted to overcome the above-mentioned limitations of the prior art by designing an anti-transferrin receptor antibody having excellent blood-brain barrier permeability through its regulated binding affinity to the transferrin receptor.

### DISCLOSURE

### Technical Problem

It is an object of the present invention to provide an anti-human transferrin receptor antibody (anti-hTfR antibody) with improved blood-brain barrier permeability.

It is another object of the present invention to provide a multi-specific antibody produced using the anti-human transferrin receptor antibody.

It is another object of the present invention to provide a pharmaceutical composition for preventing or treating brain diseases, comprising the anti-human transferrin receptor antibody or multi-specific antibody.

### Technical Solution

To achieve the above objects, the present invention provides an anti-human transferrin receptor antibody having improved blood-brain barrier permeability.

The anti-human transferrin receptor antibody of the present invention comprises a heavy chain variable region (VH) of SEQ ID NO: 1 and a light chain variable region (VL) of SEQ ID NO: 2, and one or more of tyrosines (Y) in the amino acid sequences of SEQ ID NOs: 1 and 2 are substituted with alanine (A) or histidine (H).

In the present invention, the tyrosine may be selected from the group consisting of Y27, Y98 and Y99 in SEQ ID NO: 1, and Y94 in SEQ ID NO: 2 according to Kabat numbering.

In the present invention, the substitution may comprise one or more mutations selected from the group consisting of Y27H, Y98A and Y99H in SEQ ID NO: 1, and Y94A in SEQ ID NO: 2 according to Kabat numbering.

In the present invention, one or more drug compounds may be conjugated to the anti-human transferrin receptor antibody.

The antibody of the invention may be a humanized antibody.

The present invention also provides a multi-specific antibody using the anti-human transferrin receptor antibody.

In the present invention, the multi-specific antibody may be a multi-specific antibody comprising one or more first binding domains that bind to a human transferrin receptor (hTfR) and one or more second binding domains that bind to a target molecule, wherein the first binding domain comprises a heavy chain variable region (VH) of SEQ ID NO: 1 and a light chain variable region (VL) of SEQ ID NO: 2, and one or more of tyrosines (Y) in the amino acid sequences of SEQ ID NOs: 1 and 2 are substituted with alanine (A) or histidine (H).

In the present invention, the target molecule for the second binding domain may be chondroitin sulfate, beta-secretase 1 (BACE1), gamma-secretase, amyloid beta (Abeta), epidermal growth factor receptor (EGFR), tau, apolipoprotein E4 (ApoE4), alpha-synuclein, CD20, huntingtin protein, prion protein (PrP), leucine-rich repeat kinase 2 (LRRK2), amyloid precursor protein (APP), p75 neurotrophin receptor (p75NTR), caspase 6, or glucocerebrosidase.

In the present invention, the second binding domain may comprise a protein tyrosine phosphatase sigma (PTPsigma)-derived protein.

In the present invention, the PTPsigma-derived protein may comprise amino acid sequence positions 30 to 231 of a PTPsigma protein.

In the present invention, one more of leucines (L) in the PTPsigma-derived protein may be substituted with asparagine (N).

In the present invention, the first binding domain may be in a form selected from the group consisting of Fab, scFv, di-scFv, dsFv, and (dsFv)₂.

In the present invention, the first binding domain and the second binding domain are linked to an Fc region to constitute a multi-specific antibody.

In the present invention, one or more drug compounds may be conjugated to the multi-specific antibody.

The present invention also provides a pharmaceutical composition for preventing or treating brain diseases, comprising the multi-specific antibody.

In the present invention, the brain disease may be selected from the group consisting of Parkinson's disease, Alzheimer's disease, traumatic brain injury, stroke, Huntington's disease, amyotrophic lateral sclerosis, spinal cord injury, alcoholic cranial nerve disease, alcoholic dementia, and Wernicke-Korsakoff's syndrome.

The present invention also provides a method of preventing or treating brain diseases, comprising administering a pharmaceutical composition comprising the anti-human transferrin receptor antibody or the multi-specific antibody using the same.

### Advantageous Effects

The anti-human transferrin receptor antibody (anti-hTfR antibody) designed by the present invention may bind to a human transferrin receptor (hTfR) of the blood-brain barrier to enter the cell membrane through endocytosis, and easily exit through exocytosis due to its regulated binding affinity to the hTfR. Accordingly, the anti-hTfR antibody of the present invention may have excellent transcytosis efficiency for blood-brain barrier cells to enter the brain without remaining in blood-brain barrier cells.

Therefore, the anti-hTfR antibody of the present invention may be applied to a multi-specific antibody, a drug-antibody conjugate, and the like, to efficiently deliver therapeutic proteins or drug compounds to the brain. In addition, since combining it with a protein having excellent solubility in aqueous solution as a therapeutic protein of a multi-specific antibody allows high-dose administration, its effectiveness as a therapeutic agent for brain diseases may be further improved.

### DESCRIPTION OF DRAWINGS

FIG. 1 shows tyrosine residues important for the complex structure and interaction of an anti-human transferrin receptor antibody (anti-hTfR antibody) and a human transferrin receptor (hTfR).

FIG. 2 shows Kabat numbering of the heavy chain variable region (VH) of antibody 128.1.

FIG. 3 schematically shows the structure of the multi-specific antibody according to an exemplary embodiment of the present invention.

FIG. 4 shows the SDS-PAGE results for Fab fragments of the anti-hTfR antibody according to an embodiment of the present invention.

FIG. 5 shows Fab fragment crystals of the anti-hTfR antibody according to an embodiment of the present invention.

FIGS. 6a-6b show the tertiary structure of Fab fragment crystals of the anti-hTfR antibody.

FIGS. 7a-7b show the modeling results of complexes of anti-hTfR antibody and hTfR.

FIGS. 8a-8d show the results of measuring the binding affinity of the anti-hTfR antibody according to an embodiment of the present invention to hTfR.

FIG. 9 shows the results of measuring blood-brain barrier permeability for the VH-Y27H mutated anti-hTfR antibody according to an embodiment of the present invention.

FIG. 10 shows SDS-PAGE results for the anti-hTfR:PTPsigma bi-specific antibody according to an embodiment of the present invention.

FIG. 11 shows the results of measuring the blood-brain barrier permeability for the anti-hTfR:PTPsigma bi-specific antibody according to an embodiment of the present invention.

FIG. 12 shows the results of a neurite growth assay for the PTPsigma protein according to an embodiment of the present invention.

### BEST MODE

Unless defined otherwise, all technical and scientific terms used in the present specification have the same meaning as commonly understood by those of ordinary skill in the technical field to which the present invention pertains. In general, the nomenclature used in the present specification is those well known and commonly used in the art.

The present invention relates to an anti-transferrin receptor antibody having improved blood-brain barrier permeability.

Anti-transferrin receptor antibody (anti-TfR antibody) refers to an antibody that binds to a transferrin receptor (TfR). In the present invention, the anti-transferrin receptor antibody may be an anti-human transferrin receptor antibody (anti-hTfR antibody) that undergoes endocytosis when bound to a human transferrin receptor (human TfR or hTfR), and specifically, may be an anti-hTfR antibody in which a mutation is formed in the variable region of human antibody 128.1.

In the present invention, the term “antibody” includes immunoglobulin (Ig)-derived molecules that are immunologically reactive with specific antigen(s), and the immunoglobulin may be IgG, such as IgG1, IgG2, IgG3 and IgG4, and IgA, IgE, IgD, or IgM. In addition, the term “antibody” includes both polyclonal antibodies and monoclonal antibodies, and is meant to include forms produced by genetic engineering, such as chimeric antibodies (e.g., humanized murine antibodies) and heterologous antibodies (e.g., bi-specific antibodies).

The anti-human transferrin receptor antibody of the present invention is an antibody protein comprising a variable region, and its form may be produced by changing it depending on the purpose. The anti-human transferrin receptor antibody of the present invention may be a whole antibody having both Fab and Fc regions, antibody fragments, and recombinant antibodies thereof. For example, the antibody fragment and recombinant antibody may be in the form of Fab, scFv, di-scFv, dsFv, (dsFv)₂, and the like, or in the form of linking them to the Fc region.

In the present invention, the term “mutation” is meant to include substitutions, insertions and/or deletions of amino acid residues, and preferably, the mutation in the present invention includes substitutions of amino acid residues. Substitution of an amino acid residue is represented by the sequence of the amino acid residue present in the parent wild-type protein, the number of the amino acid residue, and the substituted amino acid residue.

In the present invention, the tertiary structure of the antigen-binding region of human antibody 128.1, which has been reported to bind to the human transferrin receptor to cause endocytosis, was identified by X-ray crystallography, and based on modeling of the structure of the complex with the receptor, tyrosine residues important for protein-protein interaction were discovered, and in particular, tyrosine residues that did not affect the antibody structure when mutated were structurally predicted. Based on this, as a result of mutating specific tyrosine residues, by confirming that the interaction between the anti-human transferrin receptor antibody and the transferrin receptor was reduced and that the transcytosis of blood-brain barrier cells was remarkably improved compared to the wild-type antibody, an anti-hTfR antibody having regulated binding affinity was developed.

The anti-hTfR antibody of the present invention may effectively penetrate the blood-brain barrier, and may provide a therapeutic agent for brain diseases having improved brain delivery effect when produced in the form of a multi-specific antibody along with a protein that promotes nerve cell growth.

The anti-human transferrin receptor antibody having improved blood-brain barrier permeability according to the present invention is characterized in that one or more of tyrosines (Y) in the heavy chain variable region (VH) and/or light chain variable region (VL) of human antibody 128.1 are substituted with other amino acids.

In the present invention, the numbering in the amino acid sequence of the variable region of the human antibody 128.1 follows Kabat numbering.

The wild type (WT) sequences of the heavy chain variable region (VH) and light chain variable region (VL) of the human antibody 128.1 may be represented by following SEQ ID NOs: 1 and 2, respectively.

128.1 VH WT
[SEQ ID NO: 1]
EVQLQQSGPELVKPGASMKISCKASGYSFTGYTMNWVKQSHGENL
EWIGRINPHNGGTDYNQKFKDKAPLTVDKSSNTAYMELLSLTSED
SAVYYCARGYYYYSLDYWGQG
128.1 VL WT
[SEQ ID NQ 2]
GQIVLTQSPAIMSASPGEKVTMTCSASSSIDYIHWYQQKSGTSPK
RWIYDTSKLASGVPARFSGSGSGTSYSLTISSMEPEDAATYYCHQ
RNSYPWTFGGGTRLEIR

The anti-human transferrin receptor antibody according to the present invention comprises a heavy chain variable region of SEQ ID NO: 1 and a light chain variable region of SEQ ID NO: 2, and may have a structure in which one or more of tyrosines (Y) in the amino acid sequences of SEQ ID NOs: 1 and 2 are substituted with alanine (A) or histidine (H).

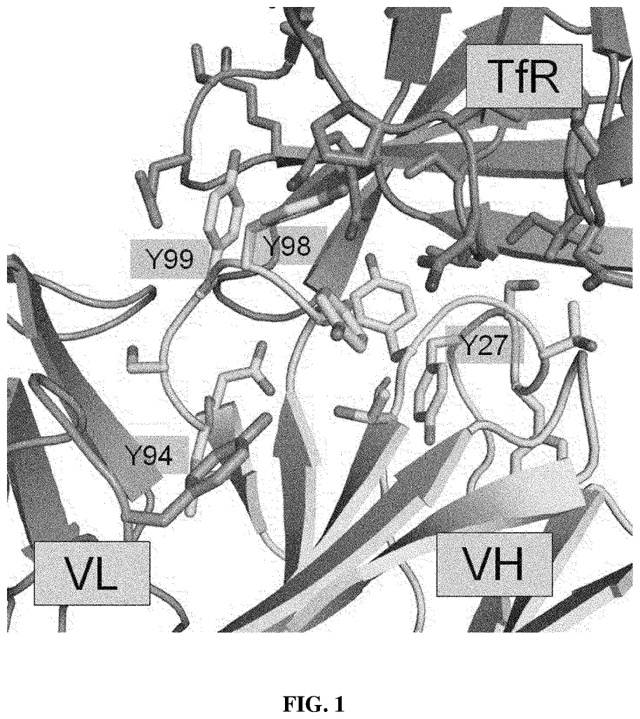

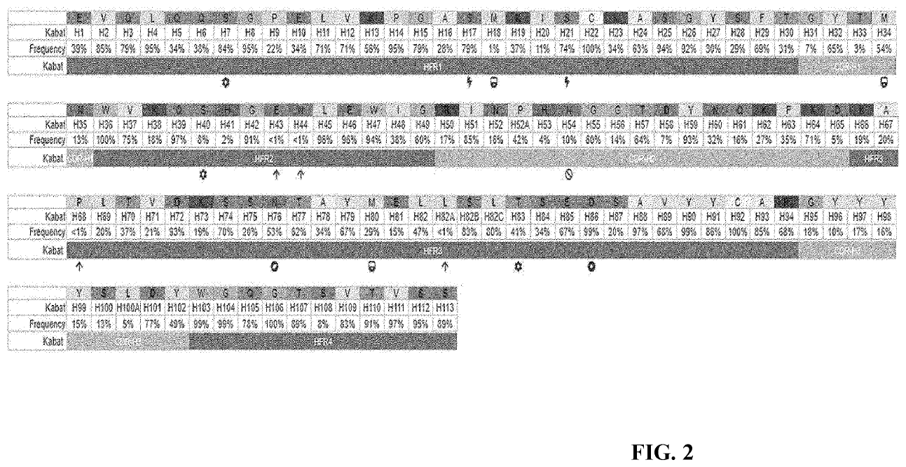

FIG. 1 shows tyrosine residues important for the complex structure and interaction of an anti-human transferrin receptor antibody and a human transferrin receptor. As shown in FIG. 1, the tyrosine to be substituted in the present invention may be one or more of Y27, Y98 and Y99 in SEQ ID NO: 1, and Y94 in SEQ ID NO: 2. FIG. 2 shows Kabat numbering of the heavy chain variable region (VH) of antibody 128.1, and with reference to this, the position of the tyrosine corresponding to the number in the heavy chain variable region of SEQ ID NO: 1 may be confirmed.

Preferably, the tyrosine may be substituted with histidine (H) or alanine (A), and the substitution may comprise one or more mutations selected from the group consisting of Y27H, Y98A and Y99H in SEQ ID NO: 1, and Y94A in SEQ ID NO: 2.

In the present invention, the anti-human transferrin receptor antibody may be a humanized antibody. That is, the anti-human transferrin receptor antibody of the present invention may comprise all or part of the amino acid sequence of the complementarity determining region (CDR) derived from a non-human animal.

According to the present invention, there may be provided an anti-human transferrin receptor antibody having improved blood-brain barrier permeability by regulating the binding affinity between the antibody and the receptor through the mutation. The anti-human transferrin receptor antibody of the present invention may be used in a multi-specific antibody or an antibody-drug conjugate to be useful as a therapeutic agent for brain diseases that requires improved blood-brain barrier permeability.

Accordingly, the present invention also provides a multi-specific antibody using the anti-human transferrin receptor antibody.

In the present invention, the term “multi-specific antibody” is meant to include an antibody that can bind to two or more different antigens or receptors, for example, a bi-specific antibody, a tri-specific antibody, and the like, and includes forms produced by genetic engineering.

In the present invention, the multi-specific antibody may comprise one or more first binding domains that bind to a human transferrin receptor (hTfR) and one or more second binding domains that bind to a target molecule. In addition, the multi-specific antibody according to the present invention may further comprise one or more binding domains (multiple binding domains) that are different from the first binding domain and the second binding domain. In this case, by binding it to other target molecules in addition to the targets of the first binding domain and the second binding domain, various treatment strategies may be proposed.

In the present invention, the term “binding domain” is interpreted as a concept that includes all antibody-derived proteins, bio-derived proteins, and artificially designed interaction proteins. For example, the second binding domain and the multiple binding domain may each independently include a bio-derived protein or an artificially designed interaction protein in addition to an antibody-derived protein.

For example, the multi-specific antibody of the invention may comprise a first binding domain that binds to a human transferrin receptor, a second binding domain that binds a target molecule, and an Fc region, and in this case, it may have a structure in which the first binding domain and the second binding domain are linked to the Fc region. Alternatively, a fusion protein produced to have multi-specificity using a linker rather than an Fc region-derived protein may also be included in the scope of the multi-specific antibody of the present invention.

Specifically, the multi-specific antibody of the present invention may have the variable region sequence of the anti-human transferrin receptor antibody of the present invention in one arm (first binding domain), and may comprise a therapeutic protein for treating brain diseases that may bind to a target molecule in the other arm (second binding domain). As a result, the multi-specific antibody of the present invention may bind to the human transferrin receptor through the first binding domain and easily penetrate the blood-brain barrier through transcytosis, thereby effectively delivering the therapeutic protein of the second binding domain to brain tissue.

In the present invention, the first binding domain is a region comprising a heavy chain variable region (VH) and a light chain variable region (VL), and its form may be produced by changing it depending on the purpose. In the present invention, the first binding domain may be in the form of Fab containing VH-CH1 and VL-CL, fragments thereof, and recombinant form. For example, the first binding domain may be in the form of Fab, scFv, di-scFv, dsFv, (dsFv)₂, and the like.

The multi-specific antibody of the present invention is characterized in that the heavy chain variable region (VH) of the first binding domain comprises SEQ ID NO: 1, and the light chain variable region (VL) comprises SEQ ID NO: 2, and one or more of tyrosines (Y) in the amino acid sequences of SEQ ID NOs: 1 and 2 are substituted with alanine (A) or histidine (H). Accordingly, the first binding domain may effectively induce transcytosis through the human transferrin receptor to transport the multi-specific antibody into the brain.

In the multi-specific antibody of the present invention, the second binding domain may comprise a protein that binds to a target molecule, for example, an antigen-binding region of an antibody that binds to a target molecule, or a therapeutic protein. In an embodiment of the present invention, the second binding domain may bind to a target molecule to promote nerve cell growth.

In the second binding domain, the antigen-binding region of the antibody that binds to the target molecule may refer to the part of the antibody that specifically binds to part or all of the antigen (target molecule) and that contains a region complementary to part or all of the antigen. The form of the antigen-binding region is not particularly limited, and may be in the form of sdAb, scFv, di-scFv, dsFv, (dsFv)₂, and the like, as well as Fab form.

The Fab form includes the variable region (VH) and the CH1 domain of constant region (CH) of the heavy chain, and the variable region (VL) and constant region (CL) of the light chain, and is a form in which a disulfide bond is formed between CH1 and CL. In addition, the sdAb form refers to a single-domain variable fragment and refers to one variable region domain.

Meanwhile, the scFv form refers to a single-chain variable fragment in which variable regions are connected, and refers to a recombinant domain in which the VH and VL regions are connected by a peptide linker. In addition, the di-scFv form refers to a recombination domain in which two scFvs are connected by a linker. The linker may be an appropriate linker known in the art, and may be a peptide consisting of 5 to 20 amino acids. Preferably, the linker may be composed of one or more amino acids selected from the group consisting of G, A, S, P, E, T, D and K. For example, the linker may be (GGGGX)ₙ, wherein X is preferably A or S, and n is preferably a natural number of 1 to 4.

In addition, the dsFv form is similar to scFv in that it is a disulfide-linked variable fragment in which the variable regions are connected, but refers to a recombinant domain in which the VH and VL regions are connected by a disulfide bond rather than a linker. The (dsFv)₂ form refers to a recombination domain in which two dsFvs are connected by a linker.

The target molecule for the second binding domain may be chondroitin sulfate, beta-secretase 1 (BACE1), gamma-secretase, amyloid beta (Abeta), epidermal growth factor receptor (EGFR), tau, apolipoprotein E4 (ApoE4), alpha-synuclein, CD20, huntingtin protein, prion protein (PrP), leucine-rich repeat kinase 2 (LRRK2), amyloid precursor protein (APP), p75 neurotrophin receptor (p75NTR), caspase 6, glucocerebrosidase, or the like.

In an embodiment of the present invention, the second binding domain may comprise a protein tyrosine phosphatase sigma (PTPsigma)-derived protein as a protein that promotes nerve cell growth.

PTPsigma is a transmembrane receptor type PTP, and is composed of three sites: an extracellular domain (ectodomain) site, a transmembrane site and a cytoplasmic site. Among these, the extracellular domain is composed of three Ig-like domains followed by several fibronectin domains, starting from the N-terminus. The extracellular domain site undergoes receptor-ligand interaction, and this interaction is transferred to the cytoplasmic site to control the enzyme activity of the PTP active domain.

In the present invention, the PTPsigma-derived protein may be a protein derived from an extracellular domain (ectodomain) of PTPsigma. In this case, the protein derived from the extracellular domain of PTPsigma refers to a protein containing all or part of the sequence of the extracellular domain of PTPsigma.

In particular, the protein derived from the extracellular domain of PTPsigma in the present invention is preferably a protein derived from the Ig-like domain of the extracellular domain, among which is more preferably a protein derived from the first to second Ig-like domains (Ig1-2) or the first to third Ig-like domains (Ig1-3).

PTPsigma binds to chondroitin sulfate in cranial nerves to cause signal transduction, and when this signal transduction occurs, neurite growth and nerve cell regeneration of cranial nerve cells are inhibited, and thus, it interferes with brain damage recovery in patients with brain diseases such as Alzheimer's disease, traumatic brain injury, and Parkinson's disease. Since the use of the present invention may block the signal transduction of PTPsigma by transporting the extracellular domain of PTPsigma to the brain and combining it with chondroitin sulfate, it is expected to be usefully used as a therapeutic agent for brain diseases.

In an embodiment of the present invention, the PTPsigma-derived protein may comprise sequence positions 30-231 of PTPsigma protein, as an amino acid sequence derived from Ig1-2 of the extracellular domain of PTPsigma.

Specifically, the PTPsigma-derived protein may comprise the following amino acid sequence of SEQ ID NO: 3:

PTPsigma 30-231
[SEQ ID NO: 3]
EEPPRFIKEPKDQIGVSGGVASFVCQATGDPKPRVTWNKKGKKVN
SQRFETIEFDESAGAVLRIQPLRTPRDENVYECVAQNSVGEITVH
AKLTVLREDQLPSGFPNIDMGPQLKVVERTRTATMLCAASGNPDP
EITWFKDFLPVDPSASNGRIKQLRSGALQIESSEETDQGKYECVA
TNSAGVRYSSPANLYVRVRRVA

In the present invention, a PTPsigma variant (mutant) for improving the solubility in aqueous solution of the multi-specific antibody may be formed in the PTPsigma-derived protein.

As described in the present invention, since a protein comprising an Ig-like domain does not have high solubility in aqueous solution, it is difficult to process it in high doses in animals or humans. In order to solve this problem, solubility was improved in the present invention by selecting amino acids that do not change the structure among hydrophobic amino acids exposed on the surface of the Ig-like domain of PTPsigma and substituting them with hydrophilic amino acids.

Specifically, the PTPsigma variant for improving the solubility may be one in which one or more residues of isoleucine (I), valine (V), leucine (L) and tyrosine (Y) in the PTPsigma-derived protein are substituted with alanine (A), serine (S), threonine (T), asparagine (N), glutamine (Q), aspartic acid (D), glutamic acid (E), lysine (K), arginine (R), or histidine (H), and preferably, may be one in which one or more of leucines (L) are substituted with asparagine (N).

Among them, one or more residues of 136, V104, V108, L125, L143, L145, L155, L187, Y224 and V227 in sequence positions 30-231 of PTPsigma are preferably substituted with A, S, T, N, Q, D, E, K, R, or H in terms of not affecting the structure of PTPsigma, and one or more residues of L143, L155, L187, Y224 and V227 are more preferably substituted with A or N in terms of improving the solubility. Since the mutation improves the solubility in aqueous solution of the multi-specific antibody to enable high-dose processing in the treatment of humans and animals, it is expected that the therapeutic effect may be further increased.

In the present invention, the first binding domain and the second binding domain may have a form linked to each chain of the Fc region.

In the present invention, the term “Fc region” refers to a C-terminal region including CH2 and CH3 domains (or CH2, CH3 and CH4 domains) of a constant region in the heavy chain of an immunoglobulin, and is used as a meaning encompassing the wild-type Fc region and its variant. The parent immunoglobulin of the Fc region may be IgG1, IgG2, IgG3 or IgG4, and preferably may be IgG1.

In the present invention, the Fc region may refer to a region extending from residue position 221 of a human IgG1 heavy chain to the C-terminus, or a region further including a hinge in the region. The number of amino acid residues in the Fc region follows EU numbering, which defines the number of residues in human immunoglobulin heavy chains.

In the present invention, the term “wild-type Fc region” includes an amino acid sequence that matches the amino acid sequence of the Fc region of an immunoglobulin found in nature.

In the present invention, the term “Fc region variant” includes one or more amino acid residues that are different from the wild-type Fc region, and may be abbreviated as “Fc variant.”

In the present invention, the Fc variant may have about 80% or more homology, preferably about 90% or more homology with the parent wild-type Fc region sequence.

In the present invention, each chain of the Fc region may include the sequence positions 221 to 447 of the IgG1 heavy chain. The sequence positions 221 to 447 of the heavy chain of the IgG1 may be represented by the following amino acid sequence of SEQ ID NO: 4:

IgG1 221-447
[SEQ ID NO: 4]
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK

In the present invention, each binding domain and the Fc region may be linked by 0 to 20 amino acid residues. That is, the binding domain and the Fc region may be directly linked, or linked through a linker consisting of 1 to 20 amino acids. In this case, each binding domain may be linked to the N-terminus, C-terminus or an amino acid located therebetween, of the Fc region, and preferably, may be linked to the N-terminus.

For example, the first binding domain may be linked to one to three of the four binding sites including the two N-termini and the two C-termini of the dimer of the Fc region, and the second binding domain may be linked to one or more of the remaining sites. Alternatively, it is also possible to produce and use a fusion protein having various orientations in which the first binding domain is linked together with the second binding domain through a linker.

In the present invention, a recombinant variant for forming multi-specific antibody may be formed in the Fc region.

For example, when the first binding domain and the second binding domain are respectively attached to the two chains of the Fc region dimer, the dimer may be formed by introducing a recombinant variant. The recombinant variant for forming the dimer may be formed using a knob-into-hole technology.

The knob-into-hole technology is a technology designed to form only heterodimers between heavy chains of antibody fragments. In this case, the knob is designed to have a side chain protruding from the opposite chain, and is inserted into a hole of the opposite domain. Due to this, heavy chains cannot be homodimerized by side chain collision, but can only be heterodimerized. In the present invention, one of the two chains constituting the Fc region may have a knob structure, and the other may have a hole structure, and in this case, the chain in which the knob or hole is formed is referred to as Fc-knob or Fc-knob, respectively.

The Fc-knob may be formed by substituting one or more amino acids in the chain constituting the Fc region with a large amino acid selected from the group consisting of tryptophan (W), arginine (R), phenylalanine (F) and tyrosine (Y). For example, the Fc-knob may have T366W variation formed in the sequence positions 221 to 447 of the IgG1-Fc heavy chain.

The Fc-hole may be formed by substituting one or more amino acids in the chain constituting the Fc region with a small amino acid selected from the group consisting of alanine (A), serine (S), threonine (T) and valine (V). For example, the Fc-hole may have T366S, L368A and Y407V variations formed in the sequence positions 221 to 447 of the IgG1-Fc heavy chain.

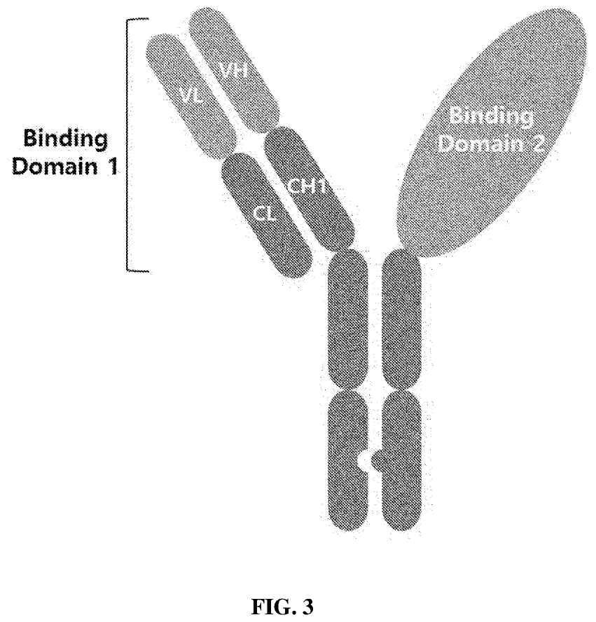

FIG. 3 schematically shows the structure of the multi-specific antibody according to an exemplary embodiment of the present invention, and the multi-specific antibody of the present invention may have a structure in which a first binding domain in the form of a Fab is linked to an Fc-hole, and a second binding domain comprising a therapeutic protein that binds to a target molecule is linked to an Fc-knob.

In an embodiment of the present invention, when the first binding domain and the second binding domain are attached to the N-terminus and C-terminus (or vice versa) of each Fc chain, the multi-specific antibody may be produced using an Fc homodimer. In this case, it may be called an Fc homodimeric multi-specific antibody.

The multi-specific antibody of the present invention may easily penetrate the blood-brain barrier through the domain that binds to the anti-human transferrin receptor antibody, and as a result, the domain that binds to the target molecule and exerts a therapeutic effect may be effectively delivered to the brain, thereby showing excellent therapeutic effects for brain diseases.

A drug compound may be conjugated to the anti-human transferrin receptor antibody or multi-specific antibody of the present invention to form an antibody-drug conjugate (ADC).

The antibody-drug conjugate is intended to deliver a small molecule drug compound to brain tissue using transcytosis of the anti-human transferrin receptor antibody through the human transferrin receptor, and when a drug compound is attached to the anti-human transferrin receptor antibody or the multispecific antibody using the same of the present invention, the delivery efficiency of drugs that are difficult to penetrate the blood-brain barrier may be improved.

In the present invention, the drug may be linked to the C-terminus and/or N-terminus of each chain of the antibody, as well as to one or more residues of amino acids within the chain. The drug may be conjugated using an appropriate linker. The drug may include drug compounds, growth inhibitors, toxins, radioisotopes, miRNA, siRNA, shRNA, and the like, as known in the art.

The present invention also relates to a pharmaceutical composition comprising the anti-human transferrin receptor antibody or the multi-specific antibody using the same of the present invention.

The pharmaceutical composition may be used to treat brain diseases, and the brain disease may comprise one or more selected from the group consisting of Parkinson's disease, Alzheimer's disease, traumatic brain injury, stroke, Huntington's disease, amyotrophic lateral sclerosis, spinal cord injury, alcoholic cranial nerve disease, alcoholic dementia, and Wernicke-Korsakoff's syndrome.

For example, when the multi-specific antibody produced by fusion with a protein that promotes nerve cell growth according to an embodiment of the present invention is used, it may be effectively used to prevent or treat brain diseases such as Parkinson's disease, Alzheimer's disease and traumatic brain injury.

The pharmaceutical composition of the present invention may comprise a pharmaceutically acceptable carrier in addition to the antibody of the present invention.

The pharmaceutically acceptable carriers are those commonly used in formulations and include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil. The pharmaceutical composition of the present invention may further comprise a lubricant, a wetting agent, a sweetener, a flavoring agent, an emulsifier, a suspending agent, a preservative, and the like, in addition to the ingredients as described above.

The pharmaceutical composition of the present invention may be administered orally or parenterally, and for parenteral administration, it may be administered by intravenous injection, subcutaneous injection, intramuscular injection, intraperitoneal injection, endothelial administration, topical administration, intranasal administration, intrapulmonary administration, intrarectal administration, and the like.

The pharmaceutical composition of the present invention may be formulated in the form of a sterile injection solution, a lyophilized formulation, a pre-filled syringe solution, an oral formulation, an external preparation or a suppository, according to conventional methods. When administered orally, the oral composition may be formulated to coat the active agent or protect it from digestion in the stomach because the protein or peptide is digested.

The pharmaceutical composition of the present invention may be formulated in a single-dose form or packaged in multi-dose vessels using a pharmaceutically acceptable carrier and/or excipient, according to methods that may be easily carried out by those skilled in the art. In this case, the formulation may be in the form of a solution in oil or aqueous medium, suspension, syrup or emulsion, or in the form of an extract, powder, powdered drug, granule, tablet or capsule, and may further comprise a dispersant or stabilizer.

The pharmaceutical composition of the present invention may further comprise at least one other therapeutic agent or diagnostic agent. For example, it may further comprise interferons, anti-S protein monoclonal antibodies, anti-S protein polyclonal antibodies, nucleoside analogs, DNA polymerase inhibitors, or siRNA preparations.

The suitable dose of the pharmaceutical composition of the present invention may be variously prescribed depending on factors such as formulation method, the mode of administration, the age, body weight, sex and pathological condition of the patient, diet, the time of administration, the route of administration, the rate of excretion, and response sensitivity. The daily dose of the pharmaceutical composition of the present invention may be 0.001 to 100 mg/kg.

The present invention also relates to a method of preventing or treating brain diseases, wherein the method may comprise administering a pharmaceutical composition comprising the anti-human transferrin receptor antibody or the multi-specific antibody using the same of the present invention.

In the present invention, the individual to be administered may be a subject, specifically, a subject in need of administration of the anti-human transferrin receptor antibody or the multi-specific antibody using the same, and the subject may be an animal, usually a mammal.

### Examples

Hereinafter, the present invention will be described in more detail through examples. However, these Examples show some experimental methods and compositions only for illustrating the present invention, and the scope of the present invention is not limited to these Examples.

### Preparative Example 1: Production of Anti-Human Transferrin Receptor Antibodies

### Gene Cloning:

The gene encoding the heavy chain (CH) and light chain (CL) of anti-human transferrin receptor antibody (anti-hTfR antibody) 128.1 was synthesized at Twist Bioscience. The variable regions of the heavy chain and light chain were amplified by polymerase chain reaction (PCR). The variable regions were inserted into the pcDNA 3.1/myc-His A plasmid vector (Invitrogen), which has a human IgG CH3-Fc region and His-tag in the heavy chain and a human kappa chain CL in the light chain.

The amino acid sequences of the variable regions (WT and mutant) of the anti-hTfR antibody used in the experiment are as follows. In the sequences below, the numbering of amino acids follows Kabat numbering.

128.1 VH WT sequence
[SEQ ID NO: 1]
EVQLQQSGPELVKPGASMKISCKASGYSFTGYTMNWVKQSHGENL
EWIGRINPHNGGTDYNQKFKDKAPLTVDKSSNTAYMELLSLTSED
SAVYYCARGYYYYSLDYWGQGTSVTVSS
128.1 VL WT sequence
[SEQ ID NO: 2]
GQIVLTQSPAIMSASPGEKVTMTCSASSSIDYIHWYQQKSGTSPK
RWIYDTSKLASGVPARFSGSGSGTSYSLTISSMEPEDAATYYCHQ
RNSYPWTFGGGTRLEIR
128.1 VH Y27H sequence
[SEQ ID NO: 5]
EVQLQQSGPELVKPGASMKISCKASGHSFTGYTMNWVKQSHGENL
EWIGRINPHNGGTDYNQKFKDKAPLTVDKSSNTAYMELLSLTSED
SAVYYCARGYYYYSLDYWGQGTSVTVSS
128.1 VH Y98A sequence
[SEQ ID NO: 6]
EVQLQQSGPELVKPGASMKISCKASGYSFTGYTMNWVKQSHGENL
EWIGRINPHNGGTDYNQKFKDKAPLTVDKSSNTAYMELLSLTSED
SAVYYCARGYYAYSLDYWGQGTSVTVSS
128.1 VH Y99H sequence
[SEQ ID NO: 7]
EVQLQQSGPELVKPGASMKISCKASGYSFTGYTMNWVKQSHGENL
EWIGRINPHNGGTDYNQKFKDKAPLTVDKSSNTAYMELLSLTSED
SAVYYCARGYYYHSLDYWGQGTSVTVSS
128.1 VL Y94A sequence
[SEQ ID NO: 8]
GQIVLTQSPAIMSASPGEKVTMTCSASSSIDYIHWYQQKSGTSPK
RWIYDTSKLASGVPARFSGSGSGTSYSLTISSMEPEDAATYYCHQ
RNSAPWTFGGGTRLEIR

### Protein Purification:

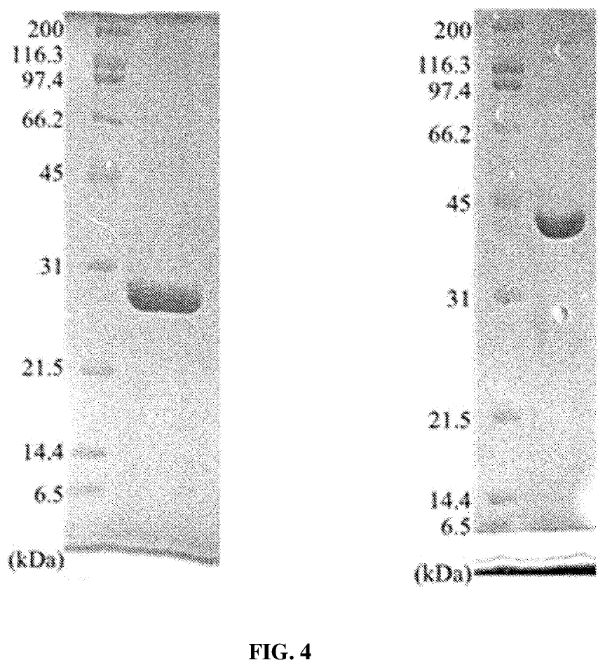

Anti-human transferrin receptor whole antibody and Fab fragments were expressed in expiCHO-S™ (Thermo Fisher Scientific). Cell culture was performed in a humidified CO₂ incubator using a 125 mL Erlenmeyer cell culture flask. To transfect the plasmid encoding the whole antibody and Fab fragments, ExpiFectamine™ CHO/plasmid DNA complexes were constructed for use. Ten days after transfection, the cell culture broth was collected, and then the protein was purified using Ni-NTA resin (QIEAGEN). SDS-PAGE was performed on the purified Fab fragments, and the results are shown in FIG. 4. In FIG. 4, the left panel is the result of the reduced condition, and the right panel is the result of the non-reduced condition.

### Experimental Example 1: Crystallization, Structure Identification and Modeling of Anti-hTfR Antibodies

### Crystallization and Structure Identification:

Fab fragment crystals of the anti-human transferrin receptor antibody were grown at 18° C. by the sitting drop vapor diffusion method. Initial crystallization was performed using a commercial screening kit, and the initial crystallization conditions were established, and then optimization was performed by changing the pH and salt concentration.

As a result, the best crystals were obtained in a reservoir solution containing 0.25 M ammonium sulfate, 0.1 M sodium acetate pH 4.5, and 31% PEG 4000. X-ray diffraction data measurements were performed using crystals cryoprotected with a solution containing 10% ethylene glycol.

X-ray diffraction experiments were performed at Pohang Accelerator beamline 11C, and diffraction was performed at 2.7 Å resolution. The results of X-ray diffraction data processing of Fab fragment crystals of the anti-human transferrin receptor antibody are shown in Table 1.

TABLE 1

| | Data collection parameters | | Results |
|---|---|---|---|
| Space group | | | P2₁2₁2₁ |
| | Cell parameters | a, b, c (Å) | 65.08, 110.76, 197.95 |
| | | α, β, γ (°) | 90.00, 119.15, 90.00 |

| | | |
|---|---|---|
| Number of reflections measured | 142786 | (7967) |
| Number of unique reflections | 35521 | (2939) |
| Resolution range (Å) | 49.49-2.75 | (2.84-2.75) |
| R-merge | 9.75 | (26.20) |
| I/σ | 11.03 | (3.22) |
| CC_{1/2} | 0.99 | (0.815) |
| Completeness (%) | 92.92 | (77.90) |
| Redundancy | 4.00 | (2.70) |

The tertiary structure was identified by molecular replacement method. Table 2 shows the refinement parameters of the tertiary structure of Fab fragment crystals of the anti-human transferrin receptor antibody.

TABLE 2

| Refinement parameters | | Results |
|---|---|---|
| Number of atoms excluding hydrogen | | 6846 |
| Classification | Protein | 6585 |
| | Non-protein | 261 |
| Protein residues | | 860 |
| R-work/R-free (%) | | 18.79/23.88 (26.64/35.70) |
| Average B-factor | | 33.43 |
| RMS | Length (Å) | 0.01 |
| | Angle (°) | 1.03 |
| Ramachandran plot | Favored (%) | 93.41 |
| | Allowed (%) | 5.29 |
| | Outliers (%) | 1.29 |

| | |
|---|---|
| Rotamer outliers (%) | 0.40 |

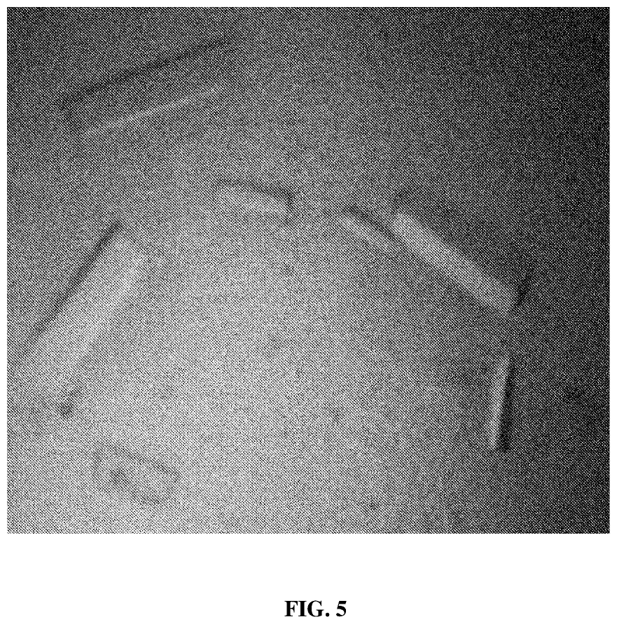

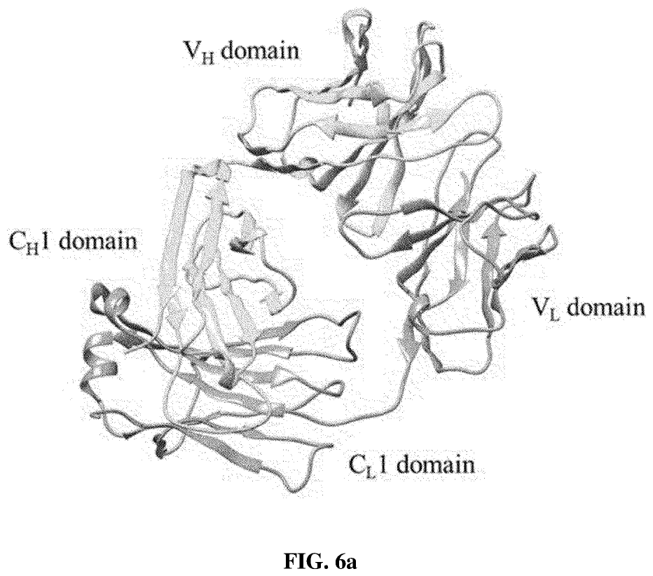

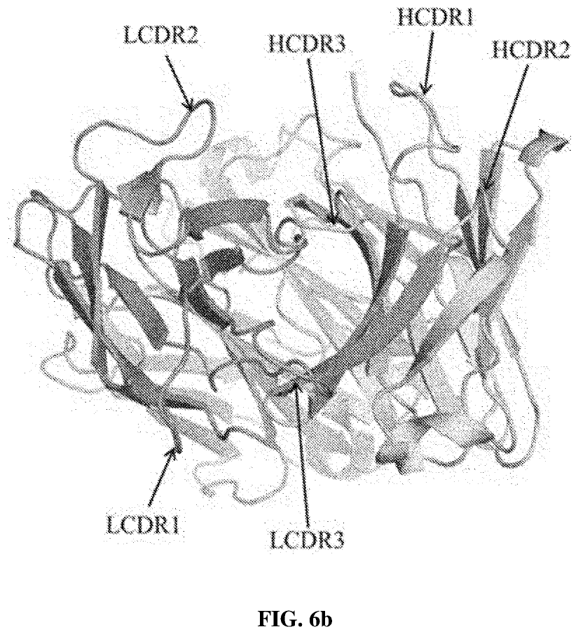

FIG. 5 is a photograph of Fab fragment crystals of the anti-human transferrin receptor antibody, and FIGS. 6a-6b show the tertiary structure of Fab fragment crystals of the anti-human transferrin receptor antibody. FIG. 6a shows the overall structure of Fab, and FIG. 6b shows the structure of the CDR loop part viewed from above.

### Modeling of Antibody-Receptor Complexes:

The complex structure of anti-human transferrin receptor antibody and human transferrin receptor (PDB id code: 3KAS) was modeled using the Patchdock program. The modeling results were validated with the Phenix program's comprehensive validation tool, MolProbity, and refinement was performed using the Firedock, fast interaction refinement program. As a result of analyzing the modeled complex structure, it was found that the tyrosine residues a shown in FIG. 1 play an important role in complex formation.

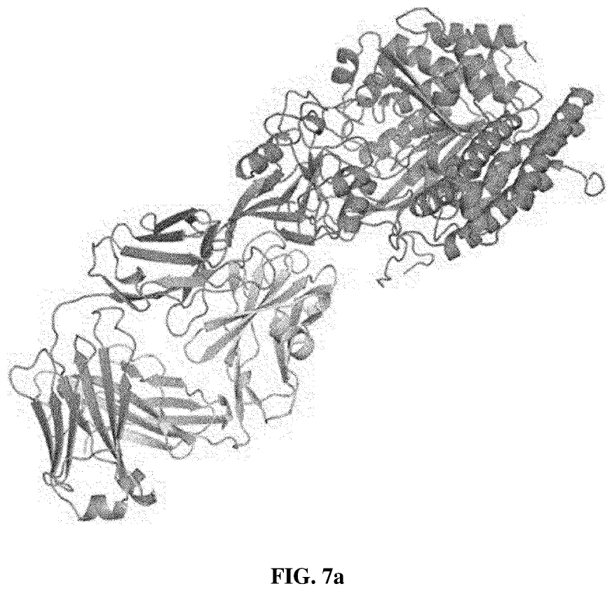

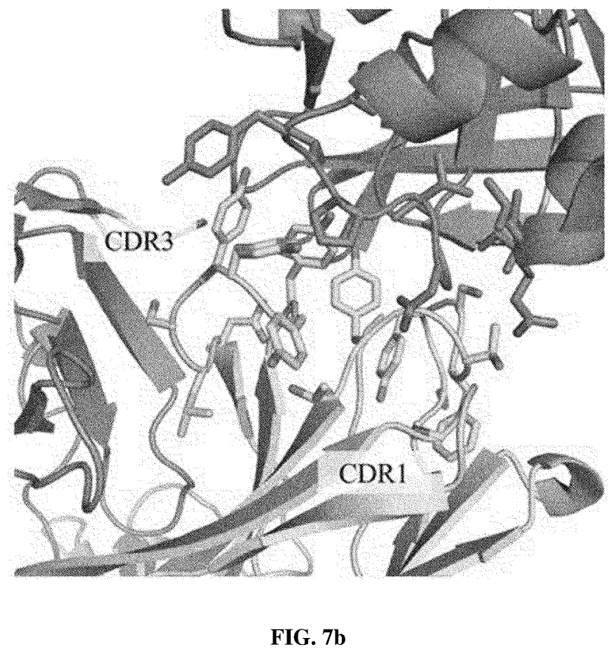

The modeling results of the complex of the Fab fragment of the anti-human transferrin receptor antibody and the human transferrin receptor are shown in FIGS. 7a-7b. FIG. 7a shows the overall structure of the complex, and FIG. 7b shows the structure of the interaction site between the antibody and receptor.

### Experimental Example 2: Measurement of Antibody Affinity

The binding affinity of the anti-human transferrin receptor antibody (anti-hTfR antibody) to the transferrin receptor (hTfR) was measured using enzyme-linked immunosorbent assay (ELISA).

Human TfR (2.5 μg/ml) was coated on a 96-well half area plate (Corning) overnight at 4° C. Antibodies were prepared by diluting 5-fold from 50 nM to 0.64 pM. PBS (pH 7.5; same as blocking buffer) containing 5% skim milk was used for dilution. The plate was washed three times with PBS at pH 7.5, and then blocked with blocking buffer at room temperature for 2 hours and washed several more times. Various concentrations of anti-TfR antibodies were placed in the wells, and binding was induced for 2 hours. Unbound antibodies were then washed twice with PBST and twice with PBS.

Horseradish peroxidase (HRP)-conjugated anti-human IgG antibody (AB frontier) was added and incubated for 2 hours. After the antibody washing step, 50 μl of TMB solution was added and incubated at 37° C. for 20 minutes. Finally, an equal volume of stop solution was added and mixed, and then the absorbance was measured at 450 nm using an EMax Microplate Reader (Molecular Devices). Steps other than TMB incubation were performed at room temperature.

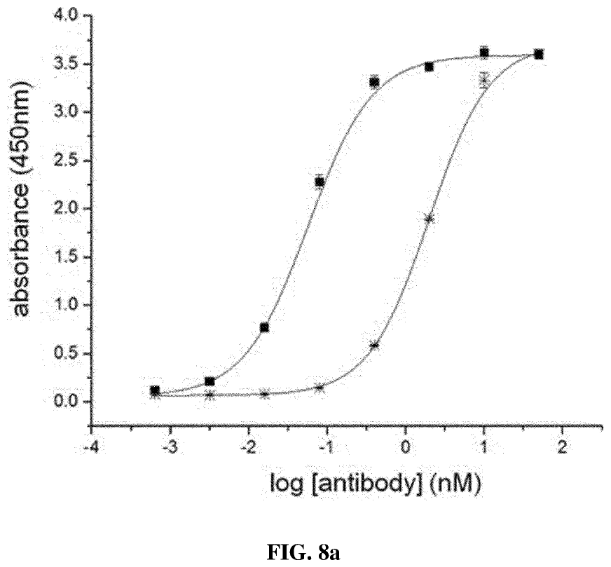

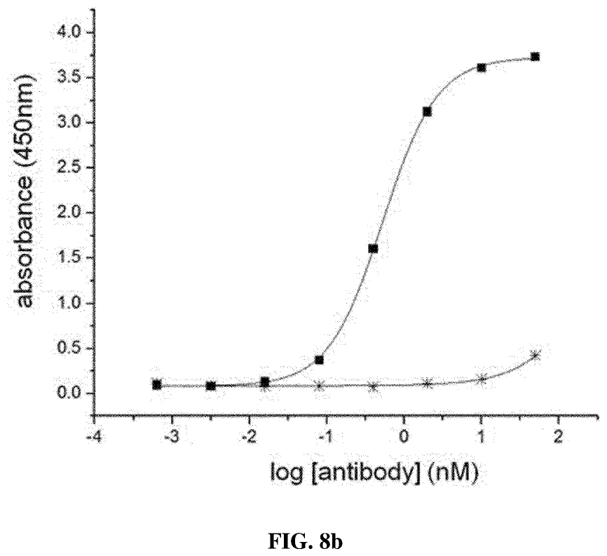

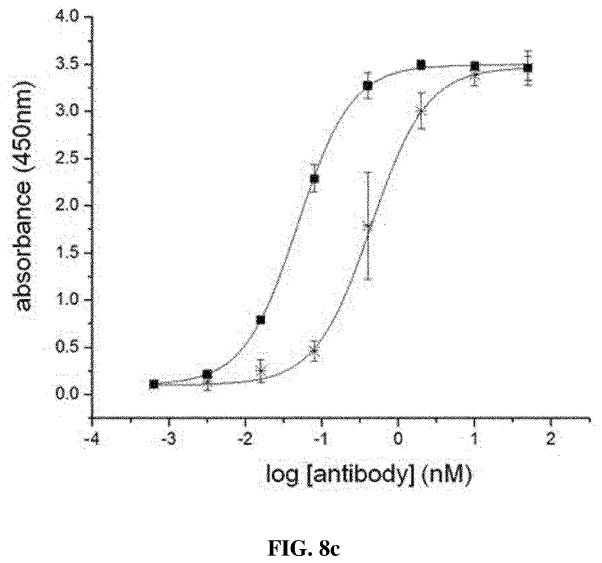

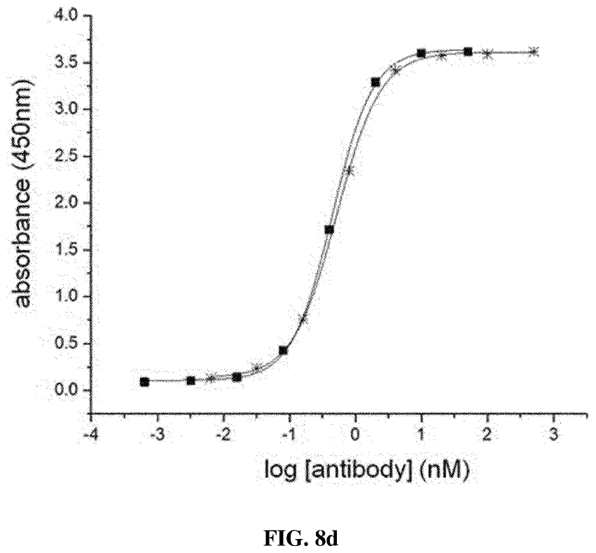

The ELISA results of measuring the binding affinity of the anti-transferrin receptor antibody designed by the present invention are shown in FIGS. 8a-8d. In the graph of FIGS. 8a-8d, the curves marked with square shapes represent the results of the wild type (WT), and the curves marked with asterisks represent the results of the mutants. FIGS. 8a-8d show the results of measuring the binding affinity for VH Y27H, VH Y98A, VH Y99H and VL Y94A, respectively.

In addition, the results of measuring the binding affinity reduction effect of the anti-transferrin receptor antibodies into which mutations were introduced are shown in Table 3.

TABLE 3

| | EC50 (nM) | Fold-increase of EC50 |
|---|---|---|
| Classification | WT/mutant | (Reduced rate of binding affinity) |
| VH Y27H | 0.057/1.98 | 35 |
| VH Y98A | 0.53/>50 | >100 |
| VH Y99H | 0.048/0.44 | 9.2 |
| VL Y94A | 0.44/0.52 | 1.2 |

From the above results, it can be confirmed that each mutant has a lower binding affinity to TfR compared to the wild type, and it was predicted that the binding affinity of VH Y27H would be suitable for optimization of blood-brain barrier permeability.

### Experimental Example 3: Blood-Brain Barrier Permeability Assay

Blood-brain barrier permeability experiments were performed using primary microvascular endothelial cells (hBMEC; Cell Systems ACBRI 376) from the human brain.

A T-75 flask was coated with Attachment Factor™ (Cell Systems). Coating was performed by placing 5 ml of Attachment Factor™ pre-warmed at 37° C. in the T-75 flask and removing it after 10 seconds. The cells were then thawed and grown to 80-90% confluence in a 5% CO₂ humidified incubator at 37° C. using complete classic medium supplemented with CultureBoost™ (Cell Systems). They were then washed with pre-warmed PBS, trypsin-EDTA was added, and incubated at 37° C. for 5 minutes. After 5 minutes, cold complete culture medium was added to inactivate trypsin. The cell suspension was transferred to a 15 ml conical tube, and centrifuged at 900×g for 10 minutes at 4° C. The upper layer of the medium was discarded, and the cells were resuspended in supplemented culture medium at 37° C.

Transwell inserts (24-well, 0.4 μm filter; SPL) were used to form an in vitro BBB model. The inserts were coated with Attachment Factor™ using the same method as described above. The obtained hBMEC cells were seeded at 20×10³ cells/well in the upper chamber of the transwell plate and incubated for 48 hours.

To confirm permeability, HRP-attached control IgG was added to the upper chamber, and after 2 hours of incubation at 37° C., the culture broths from the upper chamber and bottom chamber were recovered. The penetrated control IgG was quantitatively measured by reaction with TMB solution.

After confirming permeability, antibodies contained in fresh supplemented complete culture medium were added to the upper chamber in such a way that half of the buffer solution was replaced, and then incubated at 37° C. At 2 and 4 hours after incubation, the culture broth from the bottom chamber was recovered. Blood-brain barrier (BBB) permeability was confirmed by measuring the penetrated antibodies by sandwich ELISA using a plate coated with anti-human IgG antibody (anti-human IgG).

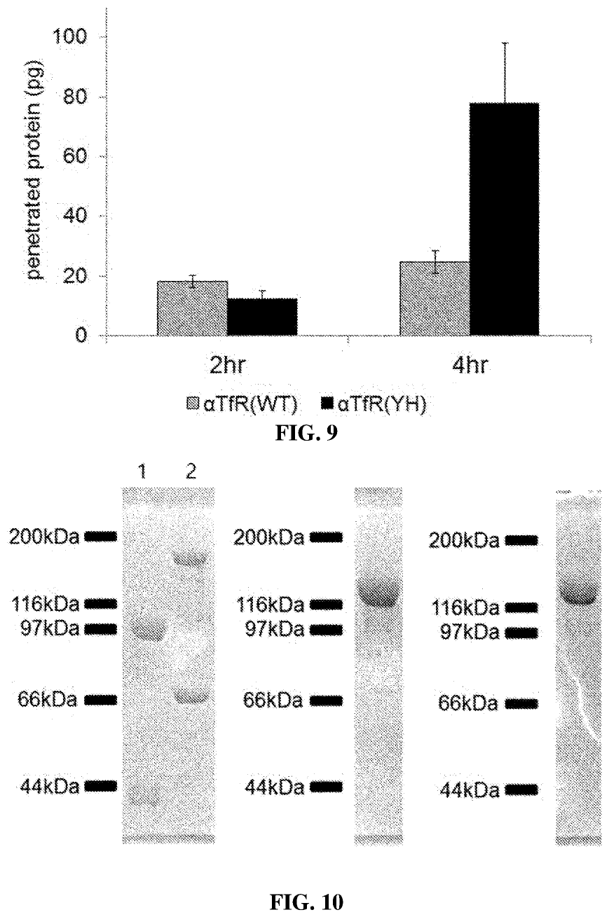

FIG. 9 shows BBB permeability of anti-transferrin receptor antibody. In FIG. 9, the permeability of 1.5 ng of wild-type anti-TfR antibody (gray) and anti-TfR antibody containing the VH-Y27H mutation (black) may be confirmed. According to the results in FIG. 9, as a result of comparing the BBB permeability of VH-Y27H among tyrosine mutants with that of the wild type, it was confirmed that BB cells were penetrated with significantly higher efficiency.

### Experimental Example 4: Blood-Brain Barrier Permeability Assay Using Bi-Specific Antibodies

### Production of Bi-Specific Antibodies Using Neuroprotective Protein:

The ectodomain Ig1-2 of PTPsigma was used as a neuroprotective protein to produce a bi-specific antibody along with an anti-transferrin receptor antibody.

Each purified protein was quantified through absorbance at 280 nm and then mixed at the same molecular ratio. Compounds were added to the mixed protein solution so that the final concentration was 0.1 M Arginine pH 9.0, 20 mM Glutathione (reduced) (Sigma-Aldrich). The reaction solution was reacted at 37° C. for 3 hours to form a bi-specific antibody.

Non-reducing SDS-PAGE was performed to confirm that the bi-specific antibody was formed in the reacted protein, and the results are shown in FIG. 10. In FIG. 10, lanes 1 and 2 of the SDS-PAGE results on the left are for PTPsigma-Fc knob and anti-TfR(WT)-HC(Fc hole), respectively, the SDS-PAGE result in the middle is for the wild-type bi-specific antibody of anti-TfR(WT):PTPsigma antibody, and the SDS-PAGE result on the right is for the bi-specific antibody anti-TfR(VH-Y27H):PTPsigma antibody, and it was confirmed through the SDS-PAGE results that the bi-specific antibodies were formed.

The amino acid sequences of anti-TfR heavy chain (HC)/light chain (LC) and PTPsigma-Fc used to produce bi-specific antibodies are as follows.

In the following anti-TfR HC/LC sequences, the underlined parts represent the sequence of the variable region, and the bold parts in HC represent the sequence of the Fc region.

[SEQ ID NO: 9 anti-TfR(WT) HC]
MDWTWRVFCLLAVAPGAHSEVQLQQSGPELVKPGASMKISCKASG
YSFTGYTMNWVKQSHGENLEWIGRINPHNGGTDYNQKFKDKAPL
TVDKSSNTAYMELLSLTSEDSAVYYCARGYYYYSLDYWGQGTSVT
VSSASTKGPSVFPLAPSSKSTSGGTAALGCLVKDYFPEPVTVSWN
SGALTSGVHTFPAVLQSSGLYSLSSVVTVPSSSLGTQTYICNVNH
KPSNTKVDKKVEPKSCDKTHTCPPCPAPELLGGPSVFLFPPKPKD
TLMISRTPEVTCVVVDVSHEDPEVKFNWYVDGVEVHNAKTKPREE
QYNSTYRVVSVLTVLHQDWLNGKEYKCKVSNKALPAPIEKTISKA
KGQPREPQVYTLPPSRDELTKNQVSLTCLVKGFYPSDIAVEWESN
GQPENNYKTTPPVLDSDGSFFLYSKLTVDKSRWQQGNVFSCSVMH
EALHNHYTQKSLSLSPGKLESRGPFEQKLISEEDLNMHTGHHHHH
H
[SEQ ID NO: 10 anti-TfR(VH Y27H) HC]
MDWTWRVFCLLAVAPGAHSEVQLQQSGPELVKPGASMKISCKASG
HSFTGYTMNWVKQSHGENLEWIGRINPHNGGTDYNQKFKDKAPLT
VDKSSNTAYMELLSLTSEDSAVYYCARGYYYYSLDYWGQGTSVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLTCLVKGFYPSDIAVEWESNG
QPENNYKTTPPVLDSDGSFFLYSKLTVDKSRWQQGNVFSCSVMHE
ALHNHYTQKSLSLSPGKLESRGPFEQKLISEEDLNMHTGHHHHHH
[SEQ ID NO: 11 anti-TfR(WT) LC]
MDFQVQIFSFLLISASVILSRGQIVLTQSPAIMSASPGEKVTMTC
SASSSIDYIHWYQQKSGTSPKRWIYDTSKLASGVPARFSGSGSG
TSYSLTISSMEPEDAATYYCHQRNSYPWTFGGGTRLEIRRTVAAP
SVFIFPPSDEQLKSGTASVVCLLNNFYPREAKVQWKVDNALQSGN
SQESVTEQDSKDSTYSLSSTLTLSKADYEKHKVYACEVTHQGLSS
PVTKSFNRGEC
[SEQ ID NO: 12 anti-TfR(WT) HC (Fc hole)]
MDWTWRVFCLLAVAPGAHSEVQLQQSGPELVKPGASMKISCKASG
YSFTGYTMNWVKQSHGENLEWIGRINPHNGGTDYNQKFKDKAPLT
VDKSSNTAYMELLSLTSEDSAVYYCARGYYYYSLDYWGQGTSVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLSCAVKGFYPSDIAVEWESNG
QPENNYKTTPPVLDSDGSFFLVSKLTVDKSRWQQGNVFSCSVMHE
ALHNHYTQKSLSLSPGKLESRGPFEQKLISEEDLNMHTGHHHHHH
[SEQ ID NO: 13 anti-TfR(VH Y27H) HC (Fc hole)]
MDWTWRVFCLLAVAPGAHSEVQLQQSGPELVKPGASMKISCKASG
HSFTGYTMNWVKQSHGENLEWIGRINPHNGGTDYNQKFKDKAPLT
VDKSSNTAYMELLSLTSEDSAVYYCARGYYYYSLDYWGQGTSVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLSCAVKGFYPSDIAVEWESNG
QPENNYKTTPPVLDSDGSFFLVSKLTVDKSRWQQGNVFSCSVMHE
ALHNHYTQKSLSLSPGKLESRGPFEQKLISEEDLNMHTGHHHHHH

In the following PTPsigma-Fc sequences, the underlined parts are the sequences of the PTPsigma-derived protein, and the bold parts are the sequences of the Fc region. In addition, the following PTPsigma (2N) sequences are the sequences comprising the L155N and L187N mutations introduced to improve the solubility in aqueous solution of the PTPsigma ectodomain Ig1-2.

[SEQ ID NO: 14 PTPsigma(WT)-Fc(WT)]
MDWTWRVFCLLAVAPGAHSDIHHHHHHEEPPRFIKEPKDQIGVSG
GVASFVCQATGDPKPRVTWNKKGKKVNSQRFETIEFDESAGAVLR
IQPLRTPRDENVYECVAQNSVGEITVHAKLTVLREDQLPSGFPNI
DMGPQLKVVERTRTATMLCAASGNPDPEITWFKDFLPVDPSASNG
RIKQLRSGALQIESSEETDQGKYECVATNSAGVRYSSPANLYVRV
RRVADKTHTCPPCPAPELLGGPSVFLFPPKPKDTLMISRTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVYTL
PPSRDELTKNQVSLTLVKGFYPSDIAVEWESNGQPENNYKTTPPV
LDSDGSFFLYSKLTVDKSRWQQGNVFSCSVMHEALHNHYTQKSLS
LSPGK
[SEQ ID NO: 15 PTPsigma(WT)-Fc(knob)]
MDWTWRVFCLLAVAPGAHSDIHHHHHHEEPPRFIKEPKDQIGVSG
GVASFVCQATGDPKPRVTWNKKGKKVNSQRFETIEFDESAGAVLR
IQPLRTPRDENVYECVAQNSVGEITVHAKLTVLREDQLPSGFPNI
DMGPQLKVVERTRTATMLCAASGNPDPEITWFKDFLPVDPSASNG
RIKQLRSGALQIESSEETDQGKYECVATNSAGVRYSSPANLYVRV
RRVADKTHTCPPCPAPELLGGPSVFLFPPKPKDTLMISRTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVYTL
PPSRDELTKNQVSLWCLVKGFYPSDIAVEWESNGQPENNYKTTPP
VLDSDGSFFLYSKLTVDKSRWQQGNVFSCSVMHEALHNHYTQKSL
SLSPGK
[SEQ ID NO: 16 PTPsigma(2N)-Fc(WT)]
MDWTWRVFCLLAVAPGAHSDIHHHHHHEEPPRFIKEPKDQIGVSG
GVASFVCQATGDPKPRVTWNKKGKKVNSQRFETIEFDESAGAVLR
IQPLRTPRDENVYECVAQNSVGEITVHAKLTVLREDQLPSGFPNI
DMGPQLKVVERTRTATMNCAASGNPDPEITWEKDFLPVDPSASNG
RIKQNRSGALQIESSEETDQGKYECVATNSAGVRYSSPANLYVRV
RRVADKTHTCPPCPAPELLGGPSVFLFPPKPKDTLMISRTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVYTL
PPSRDELTKNQVSLTCLVKGFYPSDIAVEWESNGQPENNYKTTPP
VLDSDGSFFLYSKLTVDKSRWQQGNVFSCSVMHEALHNHYTQKSL
SLSPGK
[SEQ ID NO: 17 PTPsigma(2N)-Fc(knob)]
MDWTWRVFCLLAVAPGAHSDIHHHHHHEEPPRFIKEPKDQIGVSG
GVASFVCQATGDPKPRVTWNKKGKKVNSQRFETIEFDESAGAVLR
IQPLRTPRDENVYECVAQNSVGEITVHAKLTVLREDQLPSGFPNI
DMGPQLKVVERTRTATMNCAASGNPDPEITWFKDFLPVDPSASNG
RIKQNRSGALQIESSEETDQGKYECVATNSAGVRYSSPANLYVRV
RRVADKTHTCPPCPAPELLGGPSVFLFPPKPKDTLMISRTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVYTL
PPSRDELTKNQVSLWCLVKGFYPSDIAVEWESNGQPENNYKTTPP
VLDSDGSFFLYSKLTVDKSRWQQGNVFSCSVMHEALHNHYTQKSL
SLSPGK

### Blood-Brain Barrier Permeability Assay:

Blood-brain barrier permeability experiments were performed using primary microvascular endothelial cells (hBMEC; Cell Systems ACBRI 376) from the human brain.

The cells were placed in a T-75 flask coated with Attachment Factor™ (Cell Systems) and cultured under conditions of 37° C. and 8% carbon dioxide. To perform the assay, a 24-transwell inserts from SPL Life Sciences were coated with Attachment Factor™, and then the cells isolated from the T-75 flask using trypsin were seeded at a concentration of 3×10⁴ cells/well and cultured for 5 days. To determine the formation of tight junctions, an anti-mouse IgG-HRP antibody was placed in the upper layer of the transwell and waited, and then the culture broth from the lower layer was collected, and the collected culture broth was reacted with TMB solution for 20 minutes, and used in the experiment if no color was developed.

Three antibody samples of the experimental concentration conditions were placed in the upper layer of the transwell, placed in an incubator at 37° C., and the culture broths from the lower layer of each transwell were collected at regular intervals. The concentration of antibodies in the collected culture broth was determined by ELISA assay, and the amount of penetrated antibodies was compared.

The ELISA assay was conducted using a 96-well plate. Between each step, the wells were washed four times with PBS and PBST solutions, and all antibodies were diluted in PBS containing 5% (w/v) skim milk powder. A tetramethylbenzidine substrate (LABIS KOMA) solution and a sulfuric acid solution were added, and the absorbance was measured at 450 nm.

For sample quantification in the cell permeability assay, it was coated at 4° C. with Goat Anti-Human IgG (Sigma-Aldrich) antibody first diluted to a concentration of 2 μg/mL in a sodium carbonate pH 9.5 buffer solution. After coating, the plate was blocked for 2 hours at room temperature using 5% (w/v) skim milk powder.

Antibodies to obtain a standard quantitative curve and samples obtained from the experiment were added to each prepared well and reacted at room temperature for 2 hours. To detect samples bound to the coated antibodies, they were sequentially reacted with mouse anti-myc tag 9E10 antibodies (Santa Cruz Biotechnology) and anti-mouse IgG-HRP antibodies (GWVitek) at room temperature for 2 hours, respectively.

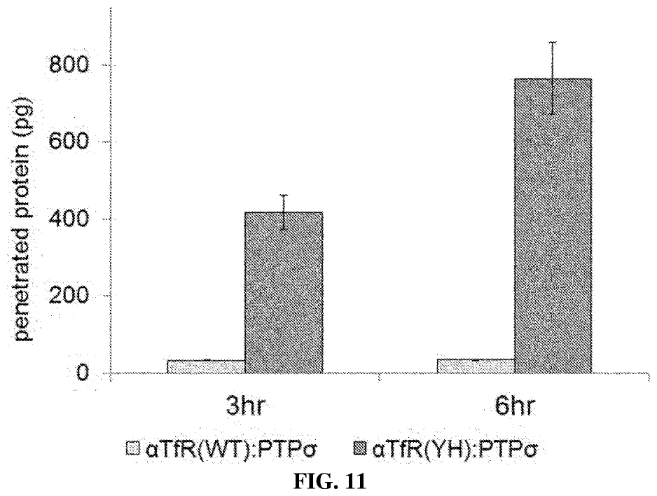

FIG. 11 is a graph showing the results of the blood-brain barrier permeability assay for the bi-specific antibody. According to FIG. 11, it was confirmed that the penetrated amount of the anti-TfR(VH-Y27H):PTPsigma antibody was much higher than that of the anti-TfR(WT):PTPsigma antibody. Accordingly, it could be confirmed that the anti-human transferrin receptor antibody produced by introducing a mutation into the variable region of human antibody 128.1 was very excellent in terms of blood-brain barrier permeability.

### Experimental Example 5: Neurite Growth Assay

Neurite outgrowth was measured using the human neuroblastoma SH-SY5Y cell line. The cells were cultured in DMEM culture medium supplemented with 10% FBS under conditions of 37° C. and 8% carbon dioxide. To perform the assay, the trypsin-isolated cells were seeded in a 96-well collagen-coated plate (SPL Life Sciences) at a concentration of 8×10³ cells/well. To induce neurite growth by differentiation, the cells were treated with 5 μM retinoic acid (Tokyo Chemical Industry), and to inhibit this, they were treated with 1 μM chondroitin sulfate sodium salt (Sigma-Aldrich).

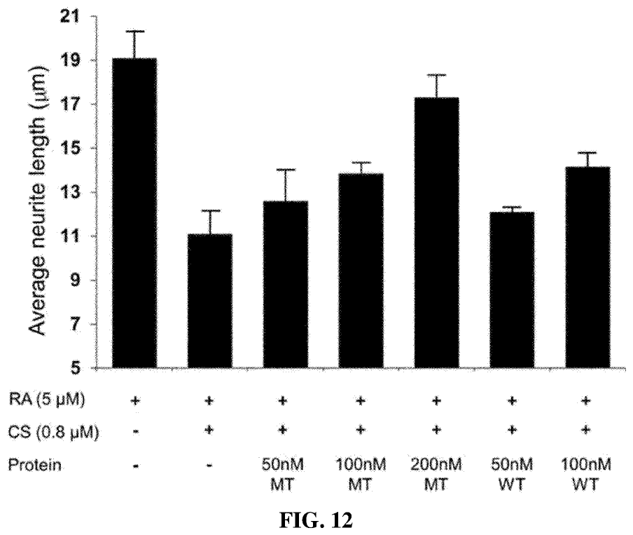

Each well was treated with the compound and PTPsigma Ig1-2 protein, and then 2 days later, measurement of neurite growth was performed by staining the cells with Neurite Stain Solution (Millipore), and the neurite growth assay results obtained by quantitatively analyzing the acquired images using the ImageJ program are shown in FIG. 12.

Referring to FIG. 12, when retinoic acid (RA) is added, neurite growth appears, and it can be seen that this growth is blocked by PTPsigma signaling by chondroitin sulfate sodium salt (CS), and in this case, it can be seen that when the PTPsigma ectodomain Ig1-2 was added, neurite growth appears again.

In FIG. 12, MT is a protein in which a 2N mutation (L155N and L187N) was introduced to improve the solubility in aqueous solution of PTPsigma ectodomain Ig1-2, and WT represents wild type PTPsigma ectodomain Ig1-2. It could be confirmed that the wild type PTPsigma ectodomain Ig1-2 protein cannot be treated at a concentration of 100 nM or more, whereas the 2N mutantion (L155N and L187N) has improved solubility and can be treated at a high concentration, thereby more effectively blocking the inhibition of neurite growth by CS.

Therefore, the bi-specific antibody produced by combining this PTPsigma mutant protein with the anti-human transferrin receptor antibody of the present invention has excellent brain injury treatment efficiency and blood-brain barrier permeability, and thus is expected to be used very effectively as a therapeutic agent for brain injury.

As a specific part of the present invention has been described in detail above, it will be apparent to those skilled in the art that these specific descriptions are only preferred embodiments, and the scope of the present invention is not limited thereby.

1. An anti-human transferrin receptor antibody comprising a heavy chain variable region (VH) of SEQ ID NO: 1 and a light chain variable region (VL) of SEQ ID NO: 2,
wherein one or more of tyrosines (Y) in the amino acid sequences of SEQ ID NOs: 1 and 2 are substituted with alanine (A) or histidine (H).

2. The anti-human transferrin receptor antibody according to claim 1, wherein the tyrosine is selected from the group consisting of Y27, Y98 and Y99 in SEQ ID NO: 1, and Y94 in SEQ ID NO: 2 according to Kabat numbering.

3. The anti-human transferrin receptor antibody according to claim 1, wherein the substitution comprises one or more mutations selected from the group consisting of Y27H, Y98A and Y99H in SEQ ID NO: 1, and Y94A in SEQ ID NO: 2 according to Kabat numbering.

4. The anti-human transferrin receptor antibody according to claim 1, wherein one or more drug compounds are conjugated to the antibody.

5. The anti-human transferrin receptor antibody according to claim 1, wherein the antibody is a humanized antibody.

6. A multi-specific antibody comprising one or more first binding domains that bind to a human transferrin receptor (hTfR) and one or more second binding domains that bind to a target molecule,
wherein the first binding domain comprises a heavy chain variable region (VH) of SEQ ID NO: 1 and a light chain variable region (VL) of SEQ ID NO: 2, and one or more of tyrosines (Y) in the amino acid sequences of SEQ ID NOs: 1 and 2 are substituted with alanine (A) or histidine (H).

7. The multi-specific antibody according to claim 6, wherein the target molecule for the second binding domain is chondroitin sulfate, beta-secretase 1 (BACE1), gamma-secretase, amyloid beta (Abeta), epidermal growth factor receptor (EGFR), tau, apolipoprotein E4 (ApoE4), alpha-synuclein, CD20, huntingtin protein, prion protein (PrP), leucine-rich repeat kinase 2 (LRRK2), amyloid precursor protein (APP), p75 neurotrophin receptor (p75NTR), caspase 6, or glucocerebrosidase.

8. The multi-specific antibody according to claim 6, wherein the second binding domain comprises a protein tyrosine phosphatase sigma (PTPsigma)-derived protein.

9. The multi-specific antibody according to claim 8, wherein the PTPsigma-derived protein comprises amino acid sequence positions 30 to 231 of a PTPsigma protein.

10. The multi-specific antibody according to claim 8, wherein one more of leucines (L) in the PTPsigma-derived protein are substituted with asparagine (N).

11. The multi-specific antibody according to claim 6, wherein the first binding domain is in a form selected from the group consisting of Fab, scFv, di-scFv, dsFv, and (dsFv)₂.

12. The multi-specific antibody according to claim 6, wherein the first binding domain and the second binding domain are linked to an Fc region.

13. The multi-specific antibody according to claim 6, wherein one or more drug compounds are conjugated to the antibody.

14. A method of preventing or treating brain diseases, comprising administering to a subject in need thereof a composition comprising the multi-specific antibody according to claim 6 in an effective amount.

15. The method of preventing or treating brain diseases according to claim 14, wherein the brain disease is selected from the group consisting of Parkinson's disease, Alzheimer's disease, traumatic brain injury, stroke, Huntington's disease, amyotrophic lateral sclerosis, spinal cord injury, alcoholic cranial nerve disease, alcoholic dementia, and Wernicke-Korsakoff's syndrome.
